(19) United States
(12) Patent Application Publication
(10) Pub. No.: US 2023/0000885 A1
(43) Pub. Date: Jan. 5, 2023

(54) Methods of Treating Psychological and Brain Disorders

(71) Applicant: University of Maryland, Baltimore, Baltimore, MD (US)

(72) Inventor: Scott Thompson, Baltimore, MD (US)

(73) Assignee: University of Maryland, Baltimore, Baltimore, MD (US)

(21) Appl. No.: 17/940,950

(22) Filed: Sep. 8, 2022

Related U.S. Application Data
(63) Continuation of application No. 17/634,729, filed on Feb. 11, 2022.
(60) Provisional application No. 62/886,090, filed on Aug. 13, 2019.

(51) Int. Cl. A61K 31/675; A61P 25/00; A61K 31/36; A61K 31/517

(52) U.S. Cl. CPC A61K 31/675; A61P 25/00; A61K 31/36; A61K 31/517

(57) ABSTRACT

Provided herein are methods for preventing or treating a psychological disorder. A serotonin agonist or an agonist of serotonin receptors is administered separately, sequentially or simultaneously in combination with a serotonin receptor 2A antagonist.

### CROSS-REFERENCE TO RELATED APPLICATION

This international application claims the benefit of priority under 35 U.S.C. § 119 (e) of provisional application U.S. Ser. No. 62/886,090, filed Aug. 13, 2019, hereby incorporated in its entirety.

### FEDERAL FUNDING LEGEND

This invention was made with government support under Grant Number MH086828 awarded by the National Institutes of Health. The government has certain rights in the invention.

### BACKGROUND OF THE INVENTION

### Field of the Invention

The present invention relates to the field of psychiatric diseases. More particularly, the present invention relates to methods of treating psychological and brain disorders by manipulating brain serotonergic systems.

### Description of the Related Art

Psychedelic substances have been used by humans for millennia for spiritual and medicinal purposes (1). Clinical research has recently begun to provide evidence supporting their use as therapeutics for numerous neuropsychiatric disorders, including obsessive-compulsive disorder, posttraumatic stress disorder, and treatment-resistant depression (TRD) (2).

A single administration of psilocybin, for example, was recently shown to significantly improve patient-reported depression scores after 1 week, with improvements persisting for up to 6 months (3). The FDA has since given psilocybin a fast-track designation for depression and additional clinical trials are underway.

While otherwise safe (4), psilocybin-induced alterations in sensory perception and consciousness are a significant barrier to its widespread utilization, necessitating a costly 6-8 hours of full-time psychological support in an inpatient setting during administration. These barriers would be greatly reduced if the psychedelic response could be blocked without impairing the antidepressant response.

Advancement of psychedelic drugs as treatments for neuropsychiatric disorders, both in terms of drug development and increasing societal acceptance, would be facilitated by a better mechanistic understanding of their therapeutic effects. Currently, the therapeutic efficacy of psychedelics has not been sufficiently studied in well validated preclinical animal models of neuropsychiatric disorders. In particular, evidence about the critical serotonin receptors required for the beneficial actions of psilocybin is lacking.

Psilocin, the active metabolite of psilocybin, is a potent agonist at almost all 5-HTRs, with affinities ranging from 3-500 nM (5), comparable to serotonin. In humans, the intensity of psilocybin-induced perceptual changes is correlated with serotonin 2A receptor (5-HT2AR) activation (6). Blocking 5-HT2Rs with ketanserin significantly attenuates self-reported perceptual distortions (7).

There is a widespread expectation, however, that psilocybin-induced alterations in consciousness are essential to the antidepressant response (2,8). For example, acute psilocybin-induced changes in processing of negative emotional stimuli in healthy subjects are prevented by ketanserin (9). However, psilocybin might relieve depressive symptoms through rapid activation of some other critical 5-HTRs.

Human depression results from a combination of genetic susceptibility and environmental factors, such as stress. Anhedonia, the inability to experience pleasure from previously enjoyable activities, is a core symptom of depression. Like human depression, various forms of chronic stress induce an anhedonic state in rodents, which is characterized by attenuated behavioral responses to previously rewarding stimuli (10). Importantly, responses to rewarding stimuli in stressed animals are restored by compounds that have antidepressant efficacy in humans, including fast- and slow-acting compounds such as ketamine and SSRIs when administered acutely and chronically, respectively (10).

Thus, there is a need in the art improved treatments for psychological and brain diseases and disorders. The present invention fulfills this longstanding need and desire in the art.

### SUMMARY OF THE INVENTION

The present invention is directed to a method for preventing or treating a psychological disorder. The method comprises the step of administering a serotonin agonist in combination with a serotonin receptor 2 antagonist, where the agonist is administered separately, sequentially or simultaneously with the antagonist.

The present invention also is directed to another method for preventing or treating a psychological disorder. The method comprises the step of administering an agonist of serotonin receptors in combination with a serotonin receptor 2 antagonist, where the agonist is administered separately, sequentially or simultaneously with the antagonist.

### BRIEF DESCRIPTION OF THE FIGURES

FIGS. 1A-1C show restoration of hedonic behavior after chronic stress by psilocybin is unaffected by ketanserin. FIG. 1A: experimental timeline illustrating when hedonic behaviors were measured in relation to chronic multimodal stress (CMMS) and drug treatment. FIG. 1B: chronic multimodal stress significantly decreased sucrose preference (SP) compared to baseline across all treatment groups: vehicle-vehicle (gray; p=0.0012; n=12), ketanserin-vehicle (blue; p=0.0012; n=6), vehicle-psilocybin (yellow; p=0.0012; n=13), ketanserin-psilocybin (green; p=0.0012; n=7). Treatment with psilocybin (1 mg/kg, i.p.) significantly increased sucrose preference compared to values after chronic multimodal stress, whether animals were pretreated with ketanserin (2 mg/kg; p=0.041) or a vehicle control (p=0.0012). Neither injection with vehicle (p=0.075;) nor ketanserin alone (p=0.87; n=6) had a significant effect on sucrose preference following chronic multimodal stress. Three-way repeated measures ANOVA revealed a significant effect of stress (F_{2,68}=55.00, p<0.0001), and interaction of Stress x Psilocybin (F_{2,68}=4.64, p=0.013). FIG. 1C: chronic multimodal stress significantly decreased preference for female urine compared to baseline: vehicle-vehicle (p=0.013; n=6), ketanserin-vehicle (p=0.0012; n=4), vehicle-psilocybin (p=0.0012; n=5), ketanserin-psilocybin (p=0.0012; n=4). Treatment with psilocybin significantly increased preference for the scent of female urine compared to values after chronic multimodal stress whether animals were pretreated with ketanserin (p=0.0024) or vehicle (p=0.0012). Following chronic multimodal stress, neither injection with vehicle (p=0.43) nor ketanserin alone (p=0.072; n=4) had a significant effect on female urine preference. Stress significantly reduced female urine preference in all groups (F_{2,30}=43.41, p<0.0001) and a three-way repeated measures ANOVA showed a significant interaction between Stress x Psilocybin (F_{2,30}=4.26, p<0.024). The figure bars represent the group means ± SEM. The reported post-hoc comparisons were corrected with the Holm-Sidak method. *p<0.05, **p<0.005; ns, not significant.

FIGS. 2A-2D show that psilocybin strengthens hippocampal TA-CA1 synapses following chronic multimodal stress. FIG. 2A: Example field EPSPs (fEPSPs) from a single stimulation intensity from one hippocampal slice per group, recorded in Mg²⁺⁻free ACSF, after wash-in of DNQX (50 μM) and then APV (80 μM) to isolate AMPA- and NMDAR-mediated components. FIG. 2B: Mice subjected to CMMS and treated with psilocybin had higher AMPA:NMDA ratios compared to stressed mice given only vehicle (gray, n=12) or ketanserin (blue, n=7), regardless whether they were pretreated with ketanserin (green, n=7; p=0.0002) or vehicle (yellow, n=13; p=0.0003). Two-way repeated measures ANOVA showed a significant effect of psilocybin (F_{1,34}=34.79, p<0.0001). FIG. 2C: Psilocybin increased the AMPA:FV ratio of the fEPSP (two-way ANOVA: F_{1,34}=4.378, p=0.044). FIG. 2D: Treatment with psilocybin did not change the NMDA:FV ratio of the fEPSP (two-way ANOVA: F_{1,34}=2.077, p=0.16). AMPA:NMDA, AMPA:FV and NMDA:FV for each animal is shown along with group means ± SEM. *p<0.05, **p<0.005, ***p<0.0005.

FIG. 3 shows that psilocybin had no effects in resilient animals. Resilient mice were defined as those exhibiting a high sucrose preference (>65%) following 14 days chronic multimodal stress. Injection of psilocybin (1 mg/kg, ip) had no effect on sucrose preference in resilient animals (n=3, red), nor did injection of vehicle (n=7, blue). Two-way repeated measures ANOVA: F_{1,14}=0.14, p=0.72. Sucrose preference is presented as the group means ± SEM with data from individual animals superimposed.

FIGS. 4A-4B show that ketanserin injection increased locomotor activity. Animals were recorded for 90 minutes following their second injection of either psilocybin (1 mg/kg) or equivalent volumes of saline vehicle. Videos were analyzed to quantify total distance travelled (cm) and time mobile (s) for each group: vehicle-vehicle (grey, n=7), vehicle-psilocybin (yellow, n=7), ketanserin-vehicle (blue, n=6), ketanserin-psilocybin (green, n=7). FIG. 4A shows that there was no significant interaction of psilocybin x ketanserin (F_{1,23}=1.47, p=0.24) on total distance travelled, but a main effect of ketanserin (F_{1,23}=6.14, p=0.021). FIG. 4B shows that for average mobility time, there was no significant interaction between psilocybin x ketanserin (F_{1,23}=0.40, p=0.53), but a significant effect of ketanserin (F_{1,23}=4.53, p=0.044). The figure bars represent the group means ± SEM. *p<0.05.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

In one embodiment of the present invention, there is provided a method for preventing or treating a psychological disorder, comprising the step of administering a serotonin agonist in combination with a serotonin receptor 2A antagonist, wherein the agonist is administered separately, sequentially or simultaneously with the antagonist. Representative examples of useful serotonin agonists include, but are not limited to, psilocybin, psilocin, baeocystin, norbaeocystin, lisurgide, LSD, dimethyltryptamine or carboxamindotryptamine, ibogaine, 3,4-methylenedioxy-methamphetamine (MDMA) or a compound that promotes a release of serotonin or a combination thereof.

In one preferred aspect of this embodiment, the serotonin agonist is psilocybin or psilocin or a derivative thereof. Representative examples of a useful derivative of psilocybin or psilocin include, but are not limited to, [3-(2-dimethylaminoethyl)-1H-indol-4-yl] dihydrogen phosphate, 4-hydroxy-N,N-dimethyltryptamine, [3-(2-methylaminoethyl)-1H -indol-4-yl] dihydrogen phosphate, 4-hydroxy-N-methyltryptamine, [3-(aminoethyl)-1H-indol -4-yl] dihydrogen phosphate, 4-hydroxytryptamine, [3-(2-trimethylaminoethyl)-1H-indol-4-yl] dihydrogen phosphate or 4-hydroxy-N,N,N-trimethyltryptamine. In this aspect, the psilocybin or psilocin is present in the form of an extract from a mushroom and/or truffle (sclerotium). Representative examples of such useful mushrooms or truffles include, but are not limited to, ones from the genus Psilocybe, Gymnopilus, Panaeolus, Copelandia, Hypholoma, Pluteus, Inocybe, Conocybe, Panaeolina, Gerronema, Agrocybe, Galerina and/or Mycena. More preferably the mushroom or truffle is P. azurescens, P. semilanceata, P. cyanescens, P. cubensis, P. subcubensis, P. tampanensis, P. mexicana, P. atlantis, and/or P. semilanceata.

In another preferred aspect of this embodiment the compound that promotes the release of serotonin is 3,4-methylenedioxymethamphetamine or a metabolite thereof. Representative examples of a useful metabolite of 3,4-methylenedioxymethamphetamine are 3,4-methylenedioxyamphetamine (MDA), 4-hydroxy-3-methoxymethamphetamine (HMMA), 4-hydroxy-3-methoxyamphetamine (HMA), 3,4-dihydroxyamphetamine (DHA), 3,4-methylenedioxyphenylacetone (MDP2P), or 3,4-methylenedioxy-N-hydroxyamphetamine (MDOH).

In this embodiment representative examples of a useful serotonin receptor 2A antagonist include, but are not limited to, MDL-11,939, ketanserin, ritanserin, altanserin, acepromazine, mianserin, quetiapine, SB204741, SB206553, SB242084, LY272015, SB243213, Blonanserin, SB200646, RS102221, nefazodone or MDL-100,907. Particularly, a serotonin receptor 2A antagonist also may be an antagonist for or simultaneously antagonize other serotonin receptors, for example, but not limited to, serotonin receptor 2B or serotonin receptor 2C.

In this embodiment and aspects thereof representative examples of a psychological disorder which may be treated by this method of the present invention include, but are not limited to, depression, psychotic disorder, schizophrenia, schizophreniform disorder (acute schizophrenic episode), schizoaffective disorder; bipolar I disorder (mania, manic disorder, manic-depressive psychosis), bipolar II disorder, major depressive disorder with psychotic feature (psychotic depression), delusional disorders (paranoia), shared Psychotic Disorder (shared paranoia disorder), Brief Psychotic disorder (other and unspecified Reactive Psychosis), psychotic disorder not otherwise specified (unspecified psychosis), paranoid personality disorder, schizoid personality disorder, schizotypal personality disorder, anxiety disorder, panic disorder, panic attacks, agoraphobia, attention deficit syndrome, premenstrual dysphoric disorder, premenstrual syndrome, ADHD, ADD, anorexia nervosa, antisocial personality disorder, autism, addiction, avoidant personality disorder, bipolar disorder, bulimia nervosa, borderline personality disorder, catatone schizophrenia, chronic motor or vocal tic disorder, conversion disorder, cyclothymia, dependent personality disorder, delier, dementia, depersonalization disorder, depression, Dhat syndrome, dissociative amnesia, dissociative fugue, dissociative identity disorder, dissociative disorder, dissociative disorder, not otherwise specified, dysthymic disorder, Da Costa's syndrome, ephobophilia, exhibitionism, generalized anxiety disorder, grandiose delusions, hypochondria, hoarding disorder, intermittent explosive disorder, jealousy, kleptomania, Kluver-Bucy syndrome, maternity psychosis, mental retardation, monomania, Munchhausen syndrome, misophony, narcissistic personality disorder, obsessive-compulsive disorder, oniomania, organic personality disorder, phobia, paranoid personality disorder, paranoid delusions, passive-aggressive personality, pathological gambling, pathological lying, personality disorder not otherwise defined, pervasive developmental disorder, pica, pain disorder, post encephalitic syndrome, postpartum depression, posttraumatic stress disorder, psychosis, psychotic disorder due to substance use, pyromania, querulant delusions, ruminational disorder, schizophrenia, schizoaffective disorder, schizoid personality disorder, schizotypal personality disorder, separation anxiety, social phobia, somatisation disorder, somatic delusion, somatoform disorder, syndrome of Capgras, syndrome of Cotard, syndrome of Ganser, syndrome of Gilles de la Tourette, selective mutism, theatrical personality disorder, trichotillomania, or undifferentiated somatoform disorder. The serotonin agonists and the serotonin receptor 2 antagonists useful in the methods of the present invention are well known to those with ordinary skill in this art and accordingly dosages, routes of administration and forms of administration (such as pills, tablets or syrups) are all well within the skill of those in this art.

In another embodiment of the present invention, there is provided a method for preventing or treating a psychological disorder, comprising the step of administering an agonist of serotonin receptors in combination with a serotonin receptor 2 antagonist, wherein the agonist is administered separately, sequentially or simultaneously with the antagonist. Representative examples of an agonist of serotonin receptors is an agonist of serotonin receptor 1B, serotonin receptor 4, serotonin receptor 6 or serotonin receptor 7.

In one aspect representative examples of an agonist of serotonin receptor 1B include, but are not limited to, ergotamine, oxymetazoline, sumatriptan, zolmitriptan, 5-carboxamidotryptamine, CGS-12066A, CP-93,129, CP-94,253, CP-122,288, CP135,807, RU24969, vortioxetine, psilocybin, psilocin, baeocystin, norbaeocystin, lisurgide, LSD, dimethyltryptamine, carboxamindotryptamine or combinations thereof. In another aspect, representative examples of an agonist of serotonin receptor 4 include, but are not limited to, BIMU-8, Cisapride, CJ-033, ML-10302, Mosapride, Prucalopride, Renzapride, RS-67506, RS-67333, SL65.0155, Tegaserod, Zacopride, Metoclopramide, Sulpiride, psilocybin, psilocin, baeocystin, norbaeocystin, lisurgide, LSD, dimethyltryptamine, carboxamindotryptamine or combinations thereof. In yet another aspect representative examples of an agonist of serotonin receptor 6 include, but are not limited to, EMD 386088, E-6801, WAY 181187 or WAY 208466. In yet another aspect representative examples of an agonist of serotonin receptor 7 include, but are not limited to, 5-carboxamidotryptamine, 5-methoxytryptamine, 8-OH-DPAT, aripiprazole, AS-19, E-55888, E-57431, 4-(2-diphenyl) -N-(1,2,3,4-tetrahydronaphthalen-1-yl)-1-piperazinehexanamide, 4-[2-(methylthio)phenyl]-N-(1,2,3,4-tetrahydro-1-naphthalenyl)-1-piperazinehexanamide, LP-211, MSD-5a, N-methylserotonin, N-1,2,3,4-Tetrahydronaphthalen-1-yl)-4-aryl-1-piperazinehexanamides, N,N-dimethytryptamine, AGH-107, AH-494, AGH-192, psilocybin, psilocin, baeocystin, norbaeocystin, lisurgide, LSD, dimethyltryptamine, or carboxamindotryptamine or a combination thereof.

In one preferred aspect, the serotonin agonist is a derivative of psilocybin or psilocin. Representative examples of a derivative of psilocybin or psilocin include but are not limited to [3-(2-dimethylaminoethyl)-1H-indol-4-yl] dihydrogen phosphate, 4-hydroxy-N, N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, 4-hydroxytryptamine, [3-(2-trimethylaminoethyl)-1H-indol-4-yl] dihydrogen phosphate, or 4-hydroxy-N,N,N-trimethyltryptamine.

In this preferred aspect of the method, the psilocybin or psilocin is present in the form of an extract from a mushroom and/or truffle (sclerotium). The mushroom or truffle may be from the genus Psilocybe, Gymnopilus, Panaeolus, Copelandia, Hypholoma, Pluteus, Inocybe, Conocybe, Panaeolina, Gerronema, Agrocybe, Galerina and/or Mycena. More preferably the mushroom or truffle is P. azurescens, P. semilanceata, P. cyanescens, P. cubensis, P. subcubensis, P. tampanensis, P. mexicana, P. atlantis, and/or P. semilanceata.

In this embodiment representative examples of a serotonin receptor 2A antagonist include, but are not limited to, MDL-11,939, ketanserin, ritanserin, altanserin, acepromazine, mianserin, quetiapine, SB204741, SB206553, SB242084, LY272015, SB243213, Blonanserin, SB200646, RS102221, nefazodone or MDL-100,907. A serotonin receptor 2A antagonist also may be an antagonist for or simultaneously antagonize other serotonin receptors, for example, but not limited to, serotonin receptor 2B or serotonin receptor 2C.

In this embodiment and all aspects thereof representative examples of a psychological disorder are chosen from, but not limited to, depression, psychotic disorder, schizophrenia, schizophreniform disorder (acute schizophrenic episode), schizoaffective disorder; bipolar I disorder (mania, manic disorder, manic-depressive psychosis), bipolar II disorder, major depressive disorder with psychotic feature (psychotic depression), delusional disorders (paranoia), shared Psychotic Disorder (shared paranoia disorder), Brief Psychotic disorder (other and unspecified Reactive Psychosis), psychotic disorder not otherwise specified (unspecified psychosis), paranoid personality disorder, schizoid personality disorder, schizotypal personality disorder, anxiety disorder, panic disorder, panic attacks, agoraphobia, attention deficit syndrome, premenstrual dysphoric disorder, premenstrual syndrome, ADHD, ADD, anorexia nervosa, antisocial personality disorder, autism, addiction, avoidant personality disorder, bipolar disorder, bulimia nervosa, borderline personality disorder, catatone schizophrenia, chronic motor or vocal tic disorder, conversion disorder, cyclothymia, dependent personality disorder, delier, dementia, depersonalization disorder, depression, Dhat syndrome, dissociative amnesia, dissociative fugue, dissociative identity disorder, dissociative disorder, dissociative disorder, not otherwise specified, dysthymic disorder, Da Costa's syndrome, ephobophilia, exhibitionism, generalized anxiety disorder, grandiose delusions, hypochondria, hoarding disorder, intermittent explosive disorder, jealousy, kleptomania, Kluver-Bucy syndrome, maternity psychosis, mental retardation, monomania, Munchhausen syndrome, misophony, narcissistic personality disorder, obsessive-compulsive disorder, oniomania, organic personality disorder, phobia, paranoid personality disorder, paranoid delusions, passive-aggressive personality, pathological gambling, pathological lying, personality disorder not otherwise defined, pervasive developmental disorder, pica, pain disorder, post encephalitic syndrome, postpartum depression, posttraumatic stress disorder, psychosis, psychotic disorder due to substance use, pyromania, querulant delusions, ruminational disorder, schizophrenia, schizoaffective disorder, schizoid personality disorder, schizotypal personality disorder, separation anxiety, social phobia, somatisation disorder, somatic delusion, somatoform disorder, syndrome of Capgras, syndrome of Cotard, syndrome of Ganser, syndrome of Gilles de la Tourette, selective mutism, theatrical personality disorder, trichotillomania, or undifferentiated somatoform disorder.

The following examples are given for the purpose of illustrating various embodiments of the invention and are not meant to limit the present invention in any fashion.

### EXAMPLE 1

### Methods

All procedures were approved by the University of Maryland Baltimore Animal Use and Care Committee and were conducted in full accordance with the National Institutes of Health Guide for the Care and Use of Laboratory Animals.

### Animals

Two cohorts of male C57BI/6J mice were used in this study in series and were bred in-house. They were 8-weeks old at the start of the experiment, kept on a 12-hour light/dark cycle (lights on at 7 am), and provided food and water ad libitum. Animals were group housed prior to the experiment but singly housed at the onset of the behavioral and stress protocols until the end of the study. Mice were assigned to balanced experimental and control groups based on hedonic behaviors assessed after stress.

### Chronic Multimodal Stress

Chronic multimodal stress was used to induce an anhedonic-like phenotype in the animals (10). The chronic multimodal stress protocol consists of 4 hrs/day of restraint stress, in which mice were immobilized in appropriately sized plastic restraint tubes and exposed to strobe lighting and white noise to minimize habituation, for 10-14 consecutive days. Stress was initiated in the morning hours, between 9-10 am, near the onset of the animals' light cycle. Following stress, rodents were returned to their home cages and singly housed.

### Hedonic Behavior

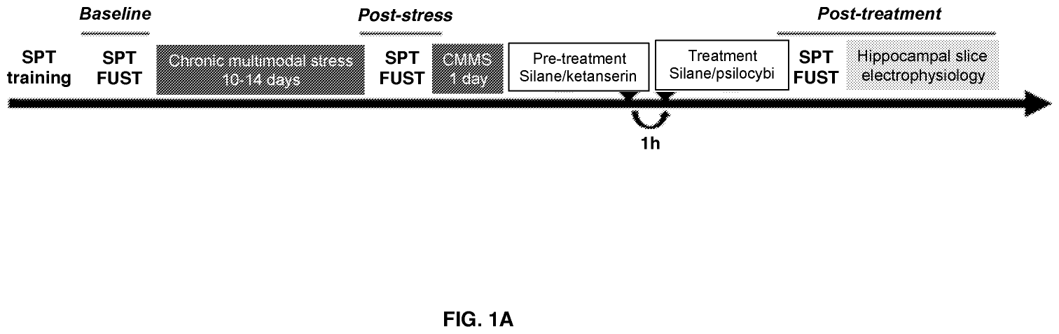

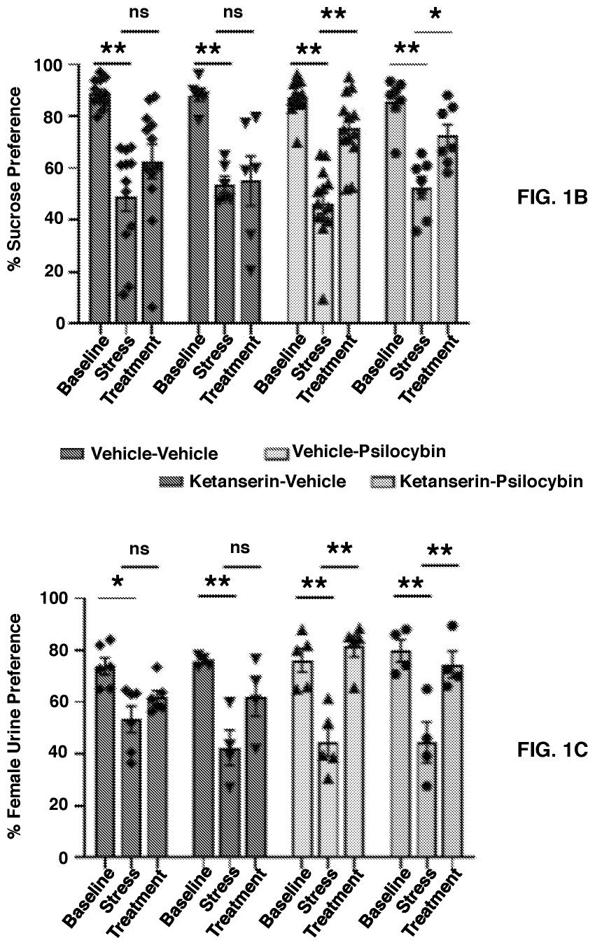

Hedonic state was assessed using the sucrose preference test (SPT) and female urine sniff test (FUST) prior to stress (baseline), after 10-14 days of chronic multimodal stress, and 24 hrs after drug injection (FIG. 1A). For the sucrose preference test, mice were exposed to a 2% sucrose solution in their home cages prior to the baseline measurement. The baseline measurements began 1 day later. On each test day, one bottle containing tap water and another bottle containing a 1% sucrose solution were placed in the cages 1-2 hours prior to the onset of the animal's dark cycle. Mice were free to consume liquid from either bottle for 14-16 hrs, after which bottles were weighed to measure consumption and replaced. The procedure was repeated for a second night with the position of the bottles reversed. Preference is expressed as a percentage and was calculated for each night as (the volume of 1% sucrose solution consumed/total liquid consumed)*100, and the preferences for the two nights were averaged.

For the female urine sniff test, mice were individually transferred to empty, freshly made cages and allowed to habituate for 15 mins. A fresh single cotton swab was then affixed to the rim of the cage, such that the tip was within reach of the mouse. One hour later, the swab was removed and replaced with 2 swabs spaced apart at the same end of the cage, one soaked in freshly collected urine from male mice and the other with urine from female mice in estrous. Video recording was started and animals were given 3 minutes to interact with the swabs. Videos were later scored by a trained experimenter blinded to the position of male and female urine swabs. Time spent sniffing each swab was recorded and percent preference scored as (time spent sniffing the female urine swab/total time spent sniffing both swabs)*100. The position of the female urine swab was switched between timepoints to account for potential side preference. As a priori criteria for inclusion in the study, mice had to have a preference for sucrose of >65% at baseline. Mice which displayed a sucrose preference of <65% following chronic multimodal stress, representing >3 standard deviations less than the historical mean baseline sucrose preference for C57 mice (89.2±6.4% (SD), n=107 animals), were considered stress susceptible. Of those mice, only those displaying a female urine preference of >65% at baseline were included in the female urine sniff test arm of the study, accounting for the differences in the reported n's. Mice having a sucrose preference >65% after 14 days of chronic multimodal stress were classified as resilient.

### Locomotion

Animals were returned to their home cages following injection with psilocybin or vehicle. A video camera was positioned overhead and animals were recorded for 90 minutes. Videos were then analyzed in Any-maze (Stoelting, Wood Dale, Ill.) to quantify distance travelled and mobility in 30 min bins for the duration of the recording. Data at 60-90 min after psilocybin or vehicle injection were normalized to the 0-30 min baseline measurements for their group.

### Electrophysiology

Standard methods were used to prepare 400 μM-thick hippocampal slices. Briefly, mice were euthanized via exposure to isoflurane followed by decapitation. Brains were excised and the hippocampus was quickly dissected from the brain and sectioned on a Leica VT1200 series vibratome in ice-cold artificial cerebrospinal fluid (ACSF) bubbled with 95% O₂/5% CO₂. The artificial cerebrospinal fluid contained: 124 mM NaCI, 3 mM KCI, 1.25 mM NaH₂PO₄, 1.5 mM MgSO₄, 2.5 mM CaCI₂, 26 mM NaHCO₃, and 10 mM glucose. Slices were allowed to recover for a minimum of 60 mins at room temperature in artificial cerebrospinal fluid in a humidified interface chamber before recording.

Use of extracellular recording, rather than whole-cell recording, was chosen for quantification of AMPA:NMDA ratios because of the complications of stress-induced changes in dendritic structure and their electrotonic influence on recordings of distal TA-CA1 synapses. For quantification of AMPA:NMDA ratios, artificial cerebrospinal fluid was prepared as stated, but without MgSO₄, to leave NMDA channels unblocked. Picrotoxin (100 μM) and CGP54626 (2 μM) were added to block GABA_{A} and GABA_{B} receptors, respectively. Slices were placed in a recording chamber and perfused with this artificial cerebrospinal fluid (1 ml/min) for the duration of the experiment. Glass recording electrodes with resistance of 3-5 MΩ were prepared and filled with recording artificial cerebrospinal fluid. These electrodes were placed in stratum lacunosum moleculare (SLM) of area CA1. Concentric bipolar tungsten electrodes were positioned in stratum lacunosum moleculare at least 500 μM from the recording electrode to stimulate temporoammonic afferents (TA). Field excitatory postsynaptic potentials (fEPSPs) were acquired using Clampex software (pClamp 10 Series, Molecular Devices), amplified (x1000, npi electronic), filtered (3 kHz), and digitized (10 kHz, Digidata 1440a, Molecular Devices). Slices were stimulated (100μs) at 0.1 Hz at five different intensities ranging from 0.01-1.0 mA, in order to collect a range of responses around a fiber volley (FV) of 0.1 mV. DNQX (50 μM) was then washed onto the slice for 15 minutes to block the AMPA component of the fEPSP and reveal the NMDA component. Five fEPSPs were again collected at the same stimulation intensities recorded prior to DNQX. The NMDAR antagonist D-APV (80 μM) was then washed onto the slice for 15 minutes to confirm that the fEPSP response remaining after DNQX was indeed NMDAR-mediated.

AMPA:NMDA ratios of the TA-CA1 fEPSPs were quantified as described previously (15) in order to provide a measure of synaptic strength across slices from different mice. All traces at each intensity were first averaged and the amplitude of FVs quantified. The AMPA component of the fEPSP was quantified as the slope over 1.5 ms at the earliest part of the linear portion of the response for each stimulation intensity, typically 0.1-2.0 ms from its initiation. The NMDA component of the fEPSP slope was quantified over 4 ms at the earliest point of the post-DNQX response fully eliminated by APV. Both AMPA and NMDA slopes were normalized to their respective FVs. For quantification, pairs of responses at the same stimulation intensities were chosen in which the response in the presence of DNQX was closest to 0.1 mV in amplitude. AMPA:NMDA ratios from each slice (1-6/mouse) were averaged to calculate each individual animal's mean AMPA:NMDA. As an independent measure of synaptic strength, we also computed AMPA:FV and NMDA:FV ratios, calculated from the same pair of responses used for AMPA:NMDA ratios. Experimenters were blind to the treatment condition during quantification and values were confirmed by a second experimenter.

### Drug Treatment

Psilocybin was obtained from Cayman Chemical (Ann Arbor, Mich.) and diluted to 1 mg/ml in sterile 0.9% saline. Ketanserin (+)-tartrate salt was purchased from MilliporeSigma (Burlington, Mass.) and also diluted to 1 mg/ml. Ketanserin was administered 60 mins prior to injection with either vehicle control or psilocybin, consistent with previous studies of ketanserin's ability to block hallucinogenic behavioral responses in humans (7) and rodents (11). Psilocybin injections were given at 1 mg/kg and ketanserin at 2 mg/kg, consistent with previous rodent studies (11, 18, 19), or equivalent volumes of saline. These doses are comparable to the oral doses used previously in human studies (psilocybin (3) ca. 0.5 mg/kg; ketanserin (11) ca. 1 mg/kg). Each experimental animal received two injections to control for any effects of injection or handling.

### Statistics

Statistical analysis consisted of Student's t-tests, two- and three-way ANOVAs using GraphPad Prism 8, with Holm-Sidak multiple comparisons corrections performed manually in Excel (Microsoft) for comparisons of interest. Results from the two cohorts of animals were not statistically different and were therefore pooled. Statistical tests used are indicated within the figure legends. Where indicated, n=number of animals.

### EXAMPLE 2

### Results

Eight week-old male C57BI/6J mice were exposed to a chronic multimodal stress paradigm and assayed hedonic state with two appetitive choice tasks involving different senses: a two-bottle sucrose preference test, comparing consumption of a 1% sucrose solution and water, and a female urine sniffing test, comparing interactions with swabs dipped in urine from male mice and female mice in estrous (FIG. 1A).

Mice displayed strong preferences for the sucrose solution and for female urine at baseline and significant decreases in both sucrose and female urine preferences after 10-14 consecutive days of chronic multimodal stress (FIG. 1B-1C). Mice were then given a single intraperitoneal (i.p.) injection of psilocybin (1 mg/kg).

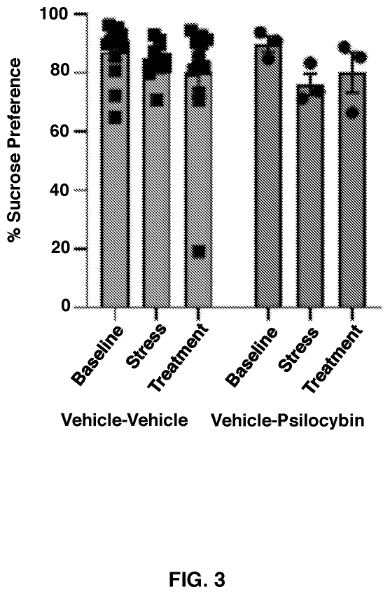

Chronic multimodal stress exposed mice displayed a significant restoration of their preference for sucrose solution and female urine 24-48 hrs after psilocybin injection, whereas mice given a saline vehicle injection retained low sucrose and female urine preferences. Stress-resilient mice that did not display loss of sucrose preference after chronic multimodal stress did not display any significant change in their responses after psilocybin injection (FIG. 3). These data represent the first evidence of a rapid anti-anhedonic response to psilocybin in a stress-induced preclinical model of depression-relevant behaviors.

Whether activation of pro-hallucinatory 5-HT2Rs was necessary for the antidepressant-like response to psilocybin was examined. The 5-HT2R antagonist ketanserin attenuates psilocybin-induced perceptual alterations in humans' and behavioral changes in rodents (11). In these same chronic multimodal stress cohorts, stress-susceptible mice received an injection of ketanserin (2 mg/kg, i.p.), followed 1 hour later by psilocybin (1 mg/kg, i.p.) or vehicle (0.9% saline), as shown to be effective in previous rodent behavioral studies (11).

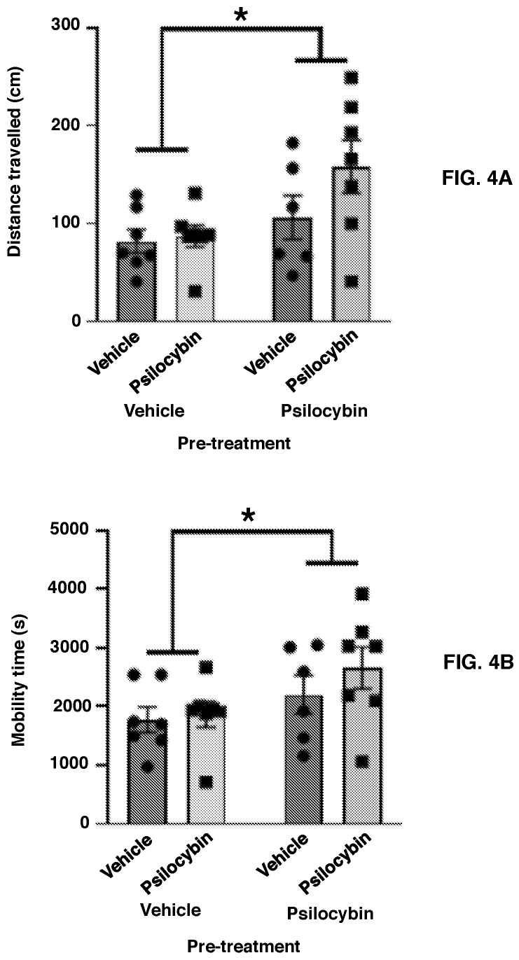

Psilocybin significantly increased sucrose and female urine preferences following stress in ketanserin-pretreated mice, (FIG. 1B-1C), whereas ketanserin pretreatment alone had no significant effect on either behavior. Ketanserin-pretreated mice displayed a significant increase in mobility, perhaps through block of 5-HT2CRs (12), regardless of whether they received psilocybin (FIG. 4), providing a positive control for ketanserin. In contrast, psilocybin at the dose used in this study had no significant effect on head twitching or mobility (11).

What mechanisms underlie the antidepressant-like behavioral response to psilocybin? A common element linking stress with the therapeutic actions of antidepressants is their shared, but opposing, effects on excitatory synapses. Chronic stress exerts deleterious effects on excitatory synaptic structure and function in multiple brain regions that are associated with cognition, reward, emotion, and motivation to work for reward (13,14). In contrast, antidepressants promote restoration of excitatory synaptic transmission in reward circuits after chronic stress (15) and restoration of functional connectivity in humans (16). There is anatomical evidence that psilocybin may promote synaptic connectivity in the prefrontal cortex via a 5-HT2R-dependent mechanism (17).

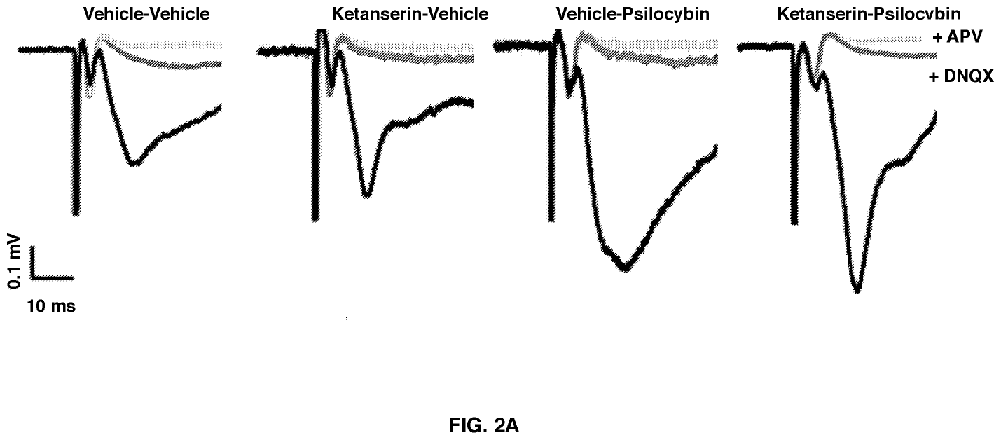

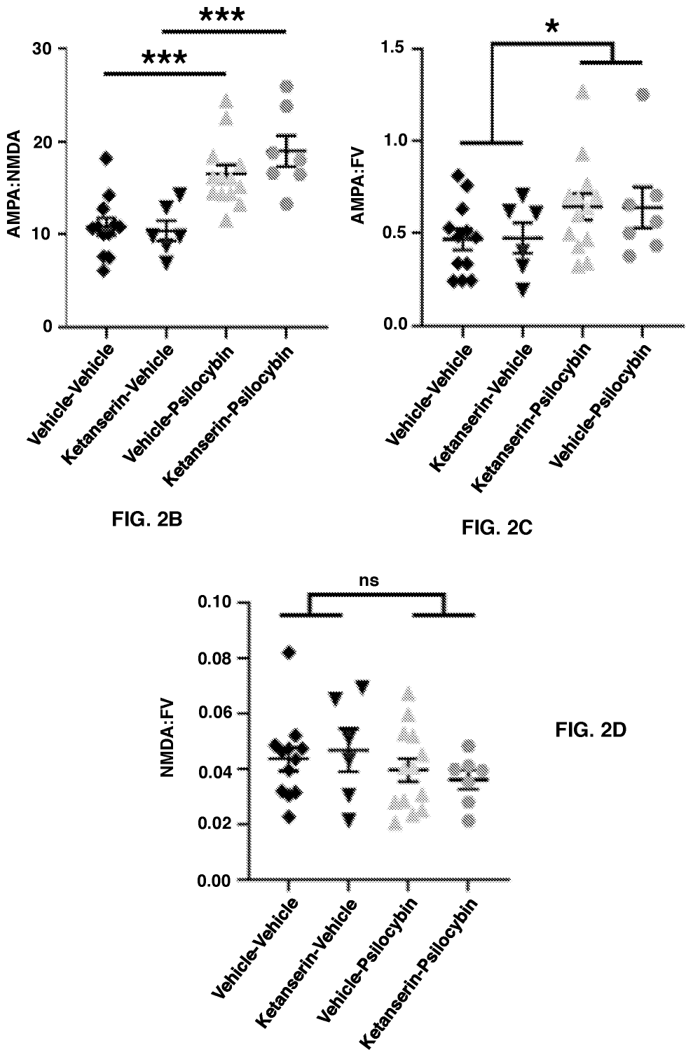

The present invention examined whether restoration of a hedonic state by psilocybin was accompanied by a restoration of excitatory synaptic strength, quantified as the ratio of the components of the field EPSP mediated by GluA (AMPA) and GluN (NMDA) receptors. After completion of the behavioral assays, hippocampal brain slices were prepared and extracellular recording was used to measure AMPA:NMDA ratios at the archetypical stress-sensitive excitatory synapse formed by temporoammonic inputs to the distal dendrites of CA1 pyramidal cells (TA-CA1; FIG. 2A) (15).

AMPA:NMDA ratios in slices taken from psilocybin-injected chronic multimodal stress-susceptible mice had AMPA:NMDA ratios were significantly greater than those in slices taken from animals subjected to chronic multimodal stress and injected with vehicle or ketanserin alone (FIG. 2B). Normalization of the individual components of the synaptic responses across slices to fiber volley amplitude revealed a greater amplitude of the AMPAR-mediated component of the response (FIG. 2C) and no significant change in the NMDAR-mediated component (FIG. 2D) in mice treated with psilocybin. These results demonstrate that a single psilocybin administration in rodents promotes persistent synaptic strengthening in a depression-relevant brain region days after its elimination from the body (hours), much like the persistent effects of psilocybin on human brain functional connectivity (18).

Consistent with the behavioral results, pretreatment with ketanserin did not impair the ability of psilocybin to restore AMPA:NMDA ratios (FIG. 2B). AMPA:NMDA ratios were significantly higher in slices taken from chronic multimodal stress animals given psilocybin compared to vehicle, regardless of whether the mice were pre-treated with ketanserin. Thus, both the anti-anhedonic behavioral response and the hippocampal synaptic response to psilocybin in mice are not dependent upon 5-HT2R activation.

The present invention provides the first evidence that psilocybin exerts a rapid beneficial action in well-studied and well-validated models of chronic stress-induced deficits in depression-relevant hedonic behaviors. Previous effects of psilocybin on behavior in the forced swim test in a selectively bred rat line have been inconsistent (18,19). Although depression is a uniquely human disease, findings from animal experiments can provide unique insights into psilocybin's mechanisms of action that are challenging to obtain in humans, such as receptor pharmacology. Indeed, these results recapitulate the rapid and persistent antidepressant actions of psilocybin in humans and the persistent alterations in brain connectivity reported previously. Although the prevailing view in developing psychedelic compounds for psychiatry is that the mind-altering effects of these compounds contribute to, or are responsible for, the therapeutic benefits (2,8), the present invention demonstrates that the 5-HT2Rs, and thus psychedelic responses, may not be required for an antidepressant response to psilocybin. Thus, the combination of psilocybin and a 5-HT2R antagonist, such as ketanserin, offers a safe and effective means to eliminate, attenuate, or shorten the duration of psilocybin-induced alterations of perception while retaining the therapeutic benefits.

Resetting synaptic strength within meso-corticolimbic circuits responsible for integration of reward and emotion, such as those demonstrated here, could provide a neurobiological substrate for lasting improvements in psychological processing. Indeed, human studies with healthy volunteers and TRD patients reveal persistent increases in resting state functional connectivity in these same circuits after psilocybin administration (20,21). The 5-HTRs underlying this response to psilocybin remain to be determined, but their definition could provide a strategy for developing alternatives to psilocybin that are biased towards synaptic strengthening relative to perceptual alteration. Previous work has suggested that 5-HT1BRs contribute to the synaptic and behavioral antidepressant-like actions of SSRIs (22). It is possible that psilocin, which has a high affinity for 5-HT1BRs (5), exerts its beneficial actions through rapid activation of 5-HT1BRs. Although animal models cannot provide insight into the potential synergistic effects of the psychedelic experience and traditional psychotherapy, in which a single administration of psilocybin may facilitate emotional insight and self-awareness, the effectiveness of these interactions may be improved with a better preclinical understanding of the pharmacological and physiological basis of psilocybin's actions.

The following references are cited herein:
1. Nichols D E. Pharmacol Rev. 68:264-35, 2016.
2. Nutt et al. Cell 181:24-28, 2020.
3. Carhart-Harris et al. Psychopharmacol. 235:399-408, 2018.
4. Johnson et al. Neuropharmacol 142:143-16, 2018.
5. Halberstadt et al. Neuropharmacology 61:364-381, 2011.
6. Madsen et al. Neuropsychopharmacol. 44:1328-133, 2019.
7. Vollenweider et al. Neuroreport 9:3897-390, 1998.
8. Roseman et al. Front. Pharmacol. 8:974, 2018.
9. Kometer M, et al. P Biol Psychiatry 72:898-90, 2012.
10. Willner P. T Neurobiol Stress. 6:78-9, 2016.
11. Winter et al. Pharmacol Biochem Behav 87:472-48, 2007.
12. Canel et al. Neuropharmacol. 70:112-121, 2013.
13. Thompson et al. Trends Neurosci. 38:279-294, 2015.
14. Duman et al. Neuron 102:75-90, 2019.
15. Kallarackal et al. J. Neurosci. 33:15669-15674, 2013.
16. Evans et al. Biol. Psychiatry 84:582-590, 2018.
17. Ly et al. Cell Reports 23:3170-3182, 2018.
18. Jefsen et al. Acta Neuropsychiatr. 31:213-219, 2019.
19. Hibicke et al. ACS Chem. Neurosci. 11:864-871, 2020.
20. Barrett et al.Sci. Rep. 10:2214, 2020.
21. Carhart-Harris et al. Sci. Rep. 7:1-11, 2017.
22. Cai et al. Nat. Neurosci. 16:464-472, 2013.
23. Belmaker R H and Agam G. N Engl J Med 358:55-68, 2008.
24. Kessler et al. JAMA 289:3095-3105, 2003.
25. Kessler et al. Arch Gen Psychiatry 62:593-602, 2005.
26. Gaynes et al. Psychiatr Serv. 60:1439-1445, 2009.
27. et al. Science 364(6436), 2019.
28. LeGates et al. Nature 564:258-262, 2018.
29. Van Dyke et al. Neuropharmacol 150:38-45, 2019.
30. Nestler et al. Neuron 34:13-25, 2002.
31. Fava et al. Neuron 28:335-341, 2000.
32. Hamet et al. Metabolism 54:0-15, 2005.
33. Billings et al. J Abnormal Psychol 92:119-133, 1983.
34. McEwen B S. Ann Rev Neurosci 22:105-122, 1999.
35. Nestler E J and Carlezon W A. Biol Psychiatry 59:1151-1159, 2006.
36. Drysdale et al. Nature Med 23:28-38, 2017.
37. Boulenguez et al. Neuropharmacol 35:1521-1529, 1996.
38. Tye et al. Nature 493:537-541, 2013.
39. Lim et al. Nature 487:183-189, 2012.
40. Yuen et al. Neuron 73:962-977, 2012.
41. Brun et al. Science 296:2243-2246, 2002.
42. Remondes M and Schuman E M. Nature 431:699-703, 2004.
43. Li et al. Nature 470:535-539, 2011.
44. Nutt et al. Lancet 376:558-1565, 2010.
45. Vollenweider F X and Kometer M. Nat Rev Neurosci 11:642-651, 2010.
46. Engel et al. Naunyn Schmiedebergs Arch Pharmacol 332:1-7, 1986.
47. Gothert M and Schlicker E. J Cardiovasc Pharmacol 10 Suppl 3:S3-S7, 1987.
48. Maura et al. Naunyn Schmiedebergs Arch Pharmacol 334:323-326, 1986.
49. Carr G V and Lucki I. Psychopharmacol (Berl) 213:265-287, 2011.
50. Svenningsson et al. Science 311:77-80, 2006.
51. Furay et al. Behav Brain Res 224:350-357, 2011.
52. Neumaier et al. Neuropsychopharmacol 15:515-522, 1996.
53. LeGates et al. Neuropsychopharmacol 44:140-154, 2019.
54. Chan et al. Neuropsychopharmacol 42:1749-1751, 2017.
55. Gerfen et al. Science 250:1429-1432, 1990.
56. Mathur et al. J Neurosci 31:7402-7411, 2011.
57. Dölen et al.Nature 501:179-184, 2013.
58. Roth et al. Crit. Rev. Neurobiol 12:319-338, 1998.
59. Lee et al. J Neurophysiol 103:479-489, 2010.
60. Hasler et al. Pharm Acta Helv 72:175-184, 1997.
61. Nautiyal et al. Neuron 86:813-826, 2015.
62. Hoyer et al. Pharmacol Rev 46:157-203, 1994.
63. Weisstaub et al. Science 313:536-540, 2006.
64. Burmeister et al. Neuropsychopharmacol. 29:660-668, 2004.
65. Burgdorf et al. J Neurosci 37:11894-11911, 2017.
66. prnewswire.com/news-releases/compass-pathways-receives-fda -breakthrough-therapy-designation-for-psilocybin-therapy-for-treatment-resistant-depression-834088100.

1-24. (Cancelled)

25. A method for treating depression or symptoms thereof in a subject in need thereof, comprising:
(a) administering to the subject a serotonin receptor 2A antagonist; and
(b) administering to the subject a serotonin agonist selected from psilocybin, psilocin, LSD and lisurgide;
wherein the serotonin agonist is administered separately, sequentially or simultaneously with the serotonin receptor 2A antagonist.

26. The method of claim 25, wherein the serotonin agonist is administered separately with the serotonin receptor 2A antagonist.

27. The method of claim 25, wherein the serotonin agonist is administered sequentially with the serotonin receptor 2A antagonist.

28. The method of claim 27, wherein the serotonin agonist is administered after the administration of the serotonin receptor 2A antagonist.

29. The method of claim 25, wherein the serotonin agonist is administered simultaneously with the serotonin receptor 2A antagonist.

30. The method of claim 25, wherein the serotonin agonist is psilocybin or psilocin.

31. The method of claim 30, wherein the serotonin agonist is psilocybin.

32. The method of claim 30, wherein the serotonin agonist is psilocin.

33. The method of claim 30, wherein the psilocybin or psilocin is present in the form of an extract from a mushroom or a truffle.

34. The method of claim 33, wherein the mushroom or truffle is from the genus Psilocybe, Gymnopilus, Panaeolus, Copelandia, Hypholoma, Pluteus, Inocybe, Conocybe, Panaeolina, Gerronema, Agrocybe, Galerina and/or Mycena.

35. The method of claim 33, wherein the mushroom or truffle is P. azurescens, P. semilanceata, P. cyanescens, P. cubensis, P. subcubensis, P. tampanensis, P. mexicana, P. atlantis, and/or P. semilanceata.

36. The method of claim 25, wherein the serotonin receptor 2A antagonist is ketanserin or ritanserin.

37. The method of claim 36, wherein the serotonin receptor 2A antagonist is ketanserin.

38. The method of claim 36, wherein the serotonin receptor 2A antagonist is ritanserin.

39. The method of claim 25, wherein the depression is major depression, psychotic depression, treatment-resistant depression (TRD), or postpartum depression.

40. The method of claim 25, wherein the administration of the serotonin receptor 2A antagonist reduces hallucinogenic effects of the serotonin agonist without impairing antidepressant effects of the serotonin agonist.

41. The method of claim 25, wherein the serotonin receptor 2A antagonist increases locomotor activity of the subject.

42. A method for treating anhedonia in a subject in need thereof, comprising:
(a) administering to the subject a serotonin receptor 2A antagonist; and
(b) administering to the subject a serotonin agonist selected from psilocybin, psilocin, LSD and lisurgide;
wherein the serotonin agonist is administered separately, sequentially or simultaneously with the serotonin receptor 2A antagonist.

43. A method for reducing suicidal ideation in a subject having depression, comprising:
(a) administering to the subject a serotonin receptor 2A antagonist; and
(b) administering to the subject a serotonin agonist selected from psilocybin, psilocin, LSD and lisurgide,
wherein the serotonin agonist is administered separately, sequentially or simultaneously with the serotonin receptor 2A antagonist.

44. A method for reducing hallucinogenic effects of a serotonin agonist in a subject, comprising administering to the subject a serotonin receptor 2A antagonist;
wherein serotonin agonist is selected from psilocybin, psilocin, LSD and lisurgide; and
the serotonin agonist is administered to the subject after the administration of the serotonin receptor 2A antagonist.
